(19) United States
(12) Patent Application Publication
(10) Pub. No.: US 2008/0312519 A1
(43) Pub. Date: Dec. 18, 2008

(54) Examination unit with an integrated mini-laboratory analysis unit

(75) Inventor: Michael Maschke, Lonnerstadt (DE)

Correspondence Address: SIEMENS CORPORATION;INTELLECTUAL PROPERTY DEPARTMENT, 170 WOOD AVENUE SOUTH, ISELIN, NJ, 08830, US

(73) Assignee: SIEMENS AKTIENGESELLSCHAFT

(21) Appl. No.: 12/157,352

(22) Filed: Jun. 10, 2008

(30) Foreign Application Priority Data
Jun. 12, 2007 (DE) 10 2007 026 910.4

(51) Int. Cl. A61B 5/05; C12Q 1/68

(52) U.S. Cl. 600/345; 435/6

(57) ABSTRACT

An examination unit, which can be used especially in the area of acute and intensive diagnostics in hospitals (e.g. in emergency rooms, intensive care units, operating theaters etc.) or in ambulances is provided. The examination unit which at least has an ultrasonic device, patient monitor, ECG, ventilator and/or resuscitation device for monitoring the vital functions and/or for the immediate medical treatment of emergency patients with symptoms of acute cardiovascular disease, myocardial infarction, angina, apoplectic stroke, etc. The device furthermore according to the invention includes an integrated mini-laboratory analysis unit for the biochemical and/or cell-biology investigation of blood samples or tissue biopsies.

### CROSS REFERENCE TO RELATED APPLICATIONS

This application claims priority of German application No. 10 2007 026 910.4 DE filed Jun. 12, 2007, which is incorporated by reference herein in its entirety.

### FIELD OF INVENTION

This invention relates to an examination unit that can particularly be used in acute and intensive diagnostics in hospitals (e.g. in emergency rooms, intensive care units, operating theaters etc.) or in ambulances, which at least has an ultrasonic device, patient monitor, ECG unit, ventilator and/or resuscitation device for monitoring the vital functions and/or for immediate medical treatment of emergency patients with symptoms of acute cardio-vascular disease, myocardial infarction, angina attack, apoplectic stroke etc. The unit according to the invention includes an integrated mini-laboratory analysis unit for the biochemical and/or cell biology investigation of blood samples, tissue biopsies etc.

### BACKGROUND OF INVENTION

Cardiovascular diseases are presently one of the most frequent causes of death in the world. Furthermore, the clinical picture of the apoplectic stroke comes second in the world as a cause of death and is the third most frequent cause of disability and premature disablement in Europe. In Germany approximately 75,000 people died of a stroke in 2003, making this disease statistically the third ranking cause of death. In the United States of America, someone has a stroke every 45 seconds. Only half the patients for whom the neurological disorders typical of a stroke occur obtain emergency hospitalization within the therapeutic time window of three hours.

In strokes, a distinction is made between the ischemic form (cerebral infarction) and the hemorrhagic form (cerebral hemorrhage). In both cases, the supply of blood to the brain is impeded causing nerve cells to die. The ischemic stroke is caused by an occlusion, i.e. by a closure of a brain artery. The latter is blocked either by a thrombus, i.e. a blood clot, or by an embolus, i.e. by a blood plug that has floated in the blood stream from a blood clot in the interior of the body of a stroke patient to the occlusion point. Approximately three quarters of all stroke patients suffer an ischemic stroke. A hemorrhagic stroke is caused by intra-cerebral bleeding where there is an egress of blood from a blood vessel into the surrounding brain tissue. In addition to the interruption to the blood supply which this causes, leading to the death of nerve cells, the accumulation of blood also increases the pressure on the brain tissue thus accelerating the death of the nerve cells.

The method of treatment of these two types of stroke varies quite considerably. In the case of an ischemic stroke, the circulation of the blood has to be encouraged, whereas with the hemorrhagic stroke the blood flow must be stemmed. With an ischaemic stroke, the most effective treatment is a thrombolytic theraphy with a recombinant tissue plasminogenic activator (rt-PA). This type of therapy would be contra-indicated in the case of a hemorrhagic stroke because with a stroke of that kind the blood is allowed to leave the brain through a puncture in order to reduce the pressure in the interior of the cranium. In the case of a hemorrhage from a burst aneurysm, the affected blood vessel must be operated on immediately. Sometimes it is possible to reduce or even stem the bleeding with medication which helps to reduce or even stops blood clotting. With today's methods of treatments and guidelines, an examination supported by CT imaging is performed after the case history has been taken and a physical examination of the stroke patient performed, in order to detect the ischemic stroke and preclude the hemorrhagic stroke.

At present there are approximately 61 million people in the USA suffering from one or more types of cardiovascular disease. Approximately 13 million people in the US have already had a myocardial infarction or suffer from angina. Also with a myocardial infarction, a quickly initiated therapy is very decisive for the survival of the patient. Normally, an initial medication with intravenous administration of nitroglycerine first takes place. Sometimes, the patient is given substances to inhibit blood clotting (anticoagulants) in the form of endogenous glycosaminolgycans such as heparin and, only in exceptional cases are special antithrombotics, such as ReoPro® (a generic designation for the glycoprotein inhibitor Abciximab) additionally used. This is usually followed by surgery where the closed coronary vessel is opened and expanded by balloon dilation.

The aforementioned examples show that with many emergency medical cases it is particularly important to arrive at a good diagnosis at the site of the emergency, in order to be able to initiate medically appropriate therapy immediately.

The treatment of a stroke normally follows according to the guidelines specific to the region or hospital. The emergency medical person in this case takes the patient's data at the site of the emergency and compiles a short case history (degree of consciousness, blood pressure, heart rate, oxygen saturation etc.), in order from this to determine the next step in the examination and treatment of the stroke patient. Based on the information obtained in this way, the suspicion that the symptoms are caused by a stroke and not due to a systemic disease (e.g. hypoglycemia) or some other neurological disease can be either confirmed or refuted. Furthermore, an initial diagnosis, which normally includes the recording of an ECG, for the exclusion of relevant cardiac dysrhythmia normally also takes place at the emergency site or in the ambulance. Where a myocardial infarction is suspected, it can be advantageous to use a 12-channel ECG to detect changes in the ST segment (the characteristic curve of the ECG in the ejection phase of the electrical systole). Where a stroke is suspected, a sonograph is of the carotid artery normally takes place to detect the presence of high-grade stenoses or occlusions.

A number of medical devices and procedures are presently available for the initial diagnosis and treatment of an acute cardiovascular disease.

Recently, the use of sonographic devices for patient diagnosis has become increasingly useful, for example in order to check the needle guidance where there is a puncture of the femoral or brachial artery. Furthermore, sonography, compared with computer tomography, offers the possibility of non-invasive medical diagnosis without exposing the patient to unnecessary radiation. Sonography can also be used for simple testing of cardiac functions.

Due to the large amount of space they require and the relatively high price, ultrasonic signaling devices are not available in all examination rooms of a hospital, and at present patients have to be sent to a special examination room for an ultrasonic examination to be performed or have to be pushed there in the hospital bed by hospital staff. Although there are at present portable sonographic devices of reduced dimensions and substantially reduced weight, e.g. the iLook® sonographic device, operated and developed by Sonosite or the portable PRIMEDIC™ HandyScan sonographic device from Metrax GmbH, Rottweil, these are, however, still so awkward to handle that they cannot be easily carried. Also there is frequently no space for an ultrasonic monitor in addition to the many life-supporting devices of an intensive care unit bed. On the other hand, the monitor for smaller sonogaphic devices is usually smaller than the palm of the hand and therefore details can no longer be usefully detected on them. Such small devices are therefore not suitable for additional use, particularly in intensive care units.

From U.S. Pat. No. 6,221,012 portable patient monitors for medical diagnosis of ECG, blood pressure and oxygen saturation are also known. Although it is possible with patient monitors of this kind to diagnose some physiological parameters at the emergency site, it is not possible to use parameters from bodily fluids, i.e. no laboratory values.

Furthermore, a combination of a patient monitor and a portable sonographic device is known from the German application paper laid open to public inspection DE 103 03 717 A1.

For a final diagnosis of the medical emergency and further therapy planning in a case of acute cardiovascular disease, other imaging methods, such as a computer tomography and laboratory tests (e.g. to investigate the blood cells, the coagulation system, certain enzymes etc.) are necessary in addition to an ultrasonic examination. In this context, laboratory equipment, such as the ADVIA® 2400 and Rapidlab® 12000 developed by Bayer are known.

Capillary electrophoresis is an established laboratory method for the investigation of the constituents of fluids. Equipment of this kind is very expensive and large. It needs between ten minutes and one hour for analysis, depending on the condition of the sample. The measuring principle is based on the fact that substances dissolved in the fluid migrate at different speeds in a capillary under the influence of an electrical field and arrive separately at the end of the measuring path. There they are detected by a detector. Software is used to calculate the relevant composition. This method provides a reliable form of diagnosis but has the disadvantage that it is possible only in a hospital.

Siemens scientist are at present working on the development of a mini-laboratory which in contrast to present laboratory analysis equipment continuously supplies data and can therefore operate as an integral part of a process. The core of this mini-laboratory, known as a “Lab-on-a-Chip” or “Quicklab” is about the size of a check card, which enables relative short analysis times of just a few minutes. In cooperation with the Institute for Spectrochemistry and Applied Spectroscopy (ISAS) in Dortmund, researchers have developed a micro fluidic system of hair-thin canals. Approximately one millionth of a liter of the fluid to be tested is ported into the analysis system each minute. Only a few millionths of a liter of this, the actual sample, are branched into a separating path a few centimeters long by switching an electrical voltage. Furthermore, the new device can also analyze several components of any samples as required.

The “Lab-on-a-Chip” project is described in, among other places, patents DE 10 2004 021 780 A1 and DE 10 2004 021 822 B3, the content of which is adopted in its entirety in this description.

Other companies, such as NANOGEN Inc., are working on solutions to realize a technical laboratory analysis of biological cell tissue and bodily fluids on a semiconductor chip.

### SUMMARY OF INVENTION

All conventional solutions, however, have the disadvantage that they only evaluate one type of parameter with one separate device in each case, so that either a number of analysis devices are necessary or they are not mobile.

Based on the prior art described above, the object of this invention is to improve the possibilities of on-site diagnosis for the initiation of suitable measures for immediate treatment of cardiovascular emergency patients at the site of the emergency, in ambulances, in the intensive care units of a hospital and at all locations at which first aid, initial diagnosis and vital life support therapies have to be provided where a cardiovascular emergency is present.

The object is achieved according to the invention by the features of the independent claims. Advantageous exemplary embodiments that expand the concept of the invention are defined in the dependent claims.

This invention relates to an examination unit with an integrated mini-laboratory analysis unit (Lab-on-a-Chip), also referred to in the following as an analysis subsystem, for the biochemical and cell-biology investigation of blood samples or tissue biopsies of a patient and with at least one device from the following group:
i. Patient monitor
ii. Ultrasonic device
iii. ECG unit
iv. Ventilator and/or
v. Resusciation unit

According to the invention, the examination unit can be designed as an emergency combi-set for the production of an initial diagnosis, monitoring vital functions and/or the initiation of emergency medical life-supporting immediate therapies for emergency patients with acute cardiovascular symptoms, myocardial infarction, stroke or diabetes mellitus symptoms. This emergency combi-set can be either a stationary or portable all-in-one ultrasonic device, patient monitor, ECG, ventilator and/or resuscitation unit (e.g. defibrillator), with the portable emergency combi-set being equipped with an integral mobile power supply unit (e.g. battery, accumulator, fuel cell or solar panel). The inventive step furthermore lies in the combination of an emergency set with a patient monitor, an imaging ultrasonic device and at least one mini-laboratory analysis unit to form an integrated unit.

The integrate mini-laboratory analysis unit of the inventive emergency combi-set furthermore has suitable technical laboratory means for performing capillary electrophoresis. With the aid of the integrated mini-laboratory analysis unit it is furthermore possible according to the invention to determine and record physiological patient parameters, including preferably ECG, blood pressure (invasive/non-invasive), respiratory rate, pulse frequency, oxygen saturation (SpO₂), CO₂ content, cardiac output and also transcutaneous O₂/CO₂). Furthermore, ultrasonic recordings of organs, bones and blood vessels can be made, displayed and stored with the aid of an ultrasonic transducer. Furthermore, it is possible with the integrated mini-laboratory analysis unit to determine and record laboratory values from biological tissue and fluids, including preferably the blood gas and blood sugar levels, the composition of the blood cells and the coagulation factors, including enzyme markers that provide evidence of a myocardial infarction: the total creatinekinase (CK-MM, CK-MB, CK-BB and CK-MiMi), the MB fraction of the creatinekinase, myoglobin (MYO), cardiac troponin I (cTnI) and cardiac troponin T (cTnT) (protein from the muscle cells of the heart that in the event of cell damage, such as during a myocardial infarction, are released into the blood) and also troponin B. These classical enzyme markers are relatively slow, i.e. they only indicate whether a cardiac infarction is present or not after a delay of several hours. Therefore, the additional analysis facilities provided by the inventive mini-laboratory analysis unit are particularly advantageous for the following cardiac markers: the long-chain heart-type fatty acid-binding protein h-FABP, sCD4OL (a biochemical marker for verification of the activation of the thrombocytes in cardiovascular diseases), myeperoxidase (MPO), the brain natriuretic peptide BNP, and its N-terminal fragment (NT-proBNP), D-dimer (a cleavage product of the fibrin), the C-reactive protein (CRP), that serves as an inflammation parameter, biopolymer markers from amino acids 2 to 13 SEQ ID No:1 (see U.S. Pat. No. 6,602,855) or the biopolymer marker ID SKITHRITHWESASLL (as described in US 2002/0160434). Furthermore, markers can also be used that indicate the presence of a stoke, such as serum phosphoglycerate mutase B type isozyme, the S-100B protein (a marker for astrocyte activities), the neurotropical growth factor of the type B, matrix-metalloproteinase-9 (MMP9), the monocyte chemotactic protein-1 (MCP-1), biopolymer markers from amino acids 2 to 13 SEQ ID No:1 (as described in U.S. Pat. No. 6,602,855), combination markers from four part markers (from the myeline protein, the β-Isoform of the S100 protein, the neuron-specific enolase and the endothelial cell membrane protein; see (DE 600 16 178 T2) or the biopolymer marker ID SKITHRITHWESASLL (as described in US 2002/0160434). Furthermore, inflammation markers that indicate arteriosclerotic plaques are used, such as the C-reactive protein (CRP) or fibrinogenic (blood-clotting factor I).

In an expanded embodiment, the integrated mini-laboratory analysis unit can also be equipped to perform gene testing using suitable technical laboratory means, in order to identify high-risk patients with a genetic predisposition to the occurrence of certain illnesses, especially a cardiovascular disease, a myocardial infarction, angina, an apoplectic stroke, arteriosclerosis or diabetes mellitus. High-risk patients in this case are those patients with which conventional risk factors such as high cholesterol, smoking, high blood pressure etc. accumulate, patients where a myocardial infarction has occurred to close family members before sixty years of age, and patients with which type II diabetes mellitus or the so called “metabolic syndrome” (a disorder of the lipid metabolism combined with overweight and high blood pressure) has been diagnosed. To determine a genetic predisposition to a myocardial infarction, the integrated mini-laboratory analysis unit can for this purpose be equipped for, amongst other things, the detection of genetic changes of the ApoB Arg3500G1n (R3500Q) and ApoB Arg3531Cys (R3531C) genes, i.e. the two most frequent mutations of the Apo B100 gene, ApoEE2/E3/E4, MTHFR Ala22Val, PAI-1 4G/5G, LPL Asn291Ser, ACE 287 bp I/D, in the intron 16, angiotensinogen Met235Thr, CETP B1/B2, Cyp 7A1-204A>C, paraoxonase 1 Gln192Arg, glycoprotein III PLA1/A2. To check a predisposition to the occurrence of a stroke, the integrated mini-laboratory analysis unit can furthermore also be equipped with suitable means for detecting genetic changes to the lysyloxidase enzyme. Furthermore, the invention can also provide that the integrated mini-laboratory analysis unit can be appropriately equipped for checking for a disposition to the occurrence of arteriosclerotic plaque. In the latter case, the integrated mini-laboratory analysis unit according to the invention has suitable means for detecting the lipoprotein-associated phospholipase A2 (Lp-LPA2 activity), which indicates an increased risk of the occurrence of a myocardial infarction or an ischemic attack, or has suitable means for detecting the lipoprotein (a) level (LP(a)) serum.

The emergency combi-set can, amongst other things, also include an ultrasonic unit (referred to in the following as an ultrasonic subsystem), which is either permanently integrated, and can thus be used only by the emergency combi-set itself, or integrated but designed as an ultrasonic unit constructed as an independent module that can be used both by the emergency combi-set itself or by external technical medical equipment.

Furthermore, the inventive emergency combi-set can have a connecting socket for a connecting cable to an external ultrasonic transducer that can be externally connected. Alternatively, a reception part for the wireless reception of ultrasonic signals from an external ultrasonic transducer via a wireless interface can be provided. The wireless interface in this case can, for example, be an infrared interface or radio interface (e.g. a Bluetooth radio path).

According to the invention, a radio signal transmission unit for the wireless transmission of recorded patient data via the air interface (e.g. via mobile radio, Bluetooth or wireless LAN) to a hospital receiving the emergency patient can also be integrated into the emergency combi-et.

A display device (referred to in the following as a patient monitor) integrated into the emergency combi-set is used to indicate acquired patient data in the form of ultrasonic recordings, measured ECG curves, data records or graphic representations of the course of other measured data. Furthermore, it is possible for the user to switch between different screen displays by means of an input menu, to change the type of display (e.g. by changing the image contrast or brightness or by zooming into a specific area of the image), or specific measured data can be displayed as numerical values. Furthermore, the user also has the facility to have several items of information as an aid to diagnosis (ECG, ultrasonic images, laboratory measured data etc.) displayed simultaneously in different display windows, in order to determine measures for the subsequent immediate therapy of the emergency patient under examination as quickly and reliably as possible from the combination of the types of information received.

Finally, it should be noted that the preceding diseases are merely examples and that this invention is not restricted to an emergency combi-set with an integrated mini-laboratory analysis unit that can be used only as a diagnostic aid for verifying these diseases. Emergency cases caused by other types of illness can also be diagnosed by a suitable configuration of the monitoring subsystem (this means the patient monitoring device), the ultrasonic subsystem and the analysis subsystem.

### BRIEF DESCRIPTION OF THE DRAWINGS

Further features of this invention are given in the dependent claims and in the description of exemplary embodiments, which are shown in the following illustrations.

FIG. 1 A schematic view of three different emergency combi-sets with an integrated mini-laboratory analysis unit and various connection possibilities for an ultrasonic transducer carried by a doctor,

FIG. 2 An illustration showing the modular construction and modular use of a mini-laboratory analysis unit integrated into such an emergency combi-set,

FIG. 3 A system overview of the system components of an emergency combi-set according to the invention with an integrated patient monitor, integrated sonographic device and integrated mini-laboratory analysis unit, and

FIG. 4 An example showing a diagnostic aid with a decision tree used for linking acquired medical information.

In the following sections, the system components of the inventive examination unit as an emergency combi-set is explained in detail with the aid of the accompanying drawings.

### DETAILED DESCRIPTION OF INVENTION

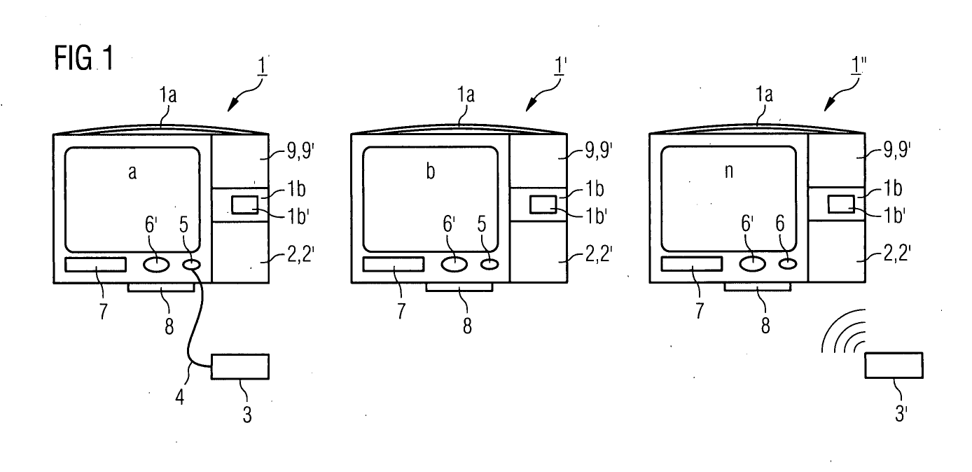

FIG. 1 is a schematic block diagram of three different emergency combi-sets 1, 1′ and 1″ according to the present invention, with an integrated mini-laboratory analysis unit 1b (with a control panel 1b′ on the front) for biochemical and/or cell-biology investigation of blood samples and/or tissue biopsies taken from a cardiovascular emergency patient. In order to be able to better transport the emergency combi-set 1, 1′ and 1″, a carrier strap or handle 1a is fitted to the top. The three emergency combi-sets 1, 1′ and 1″ are furthermore each equipped with patient monitors (a, b or n) and integrated sonographic devices 2 (with integrated control panels 2′) for processing and visualizing ultrasonic images from the interior of the body of an emergency patient with acute cardiovascular symptoms. These sonographic devices 2 furthermore serve for the processing of ultrasonic signals, via a wired (5) or wireless data interface (6) from an ultrasonic transducer 3 or 3′, which is carried by a doctor. For this purpose, in the wired exemplary embodiment in the left part of the picture of FIG. 1 a connecting cable 4 is provided with a plug part that can be plugged into a connecting socket 5 of the patient monitor of the emergency combi-set 1, in order to form a connection to the relevant patient monitor. A corresponding emergency combi-set 1′ with an integrated sonographic device, but without a connected ultrasonic transducer 3, is shown in the center part of the picture of FIG. 1. Instead of a connection via a cable 4, a wireless connection via an infrared interface or via a Bluetooth radio path can be provided, as is shown in the right part of the picture in FIG. 1. The ultrasonic transducer 3′ transmits the data by means of a built-in HF transmitter to an HF receiver part 6 integrated in to the patient monitor of the emergency combi-set 1″. The emergency combi-sets 1, 1′ and 1″ furthermore each have an integrated control module 9 with a control panel 9′ on the front for control of the respective patient monitor.

A radio signal transmission unit 6′ integrated into one of the emergency combi-sets 1, 1′ or 1″ serves, according to the invention, for the wireless transmission of recorded patient data to a hospital receiving the respective emergency patient.

An integrated mobile power supply unit in the form of a battery, an accumulator, a fuel cell or a solar panel serves as already mentioned for the power supply of the portable emergency combi-sets, 1, 1′ and 1″ For operation using accumulators, as shown in FIG. 1, the individual accumulators are mounted in a holder and charging station 8 provided for the purpose.

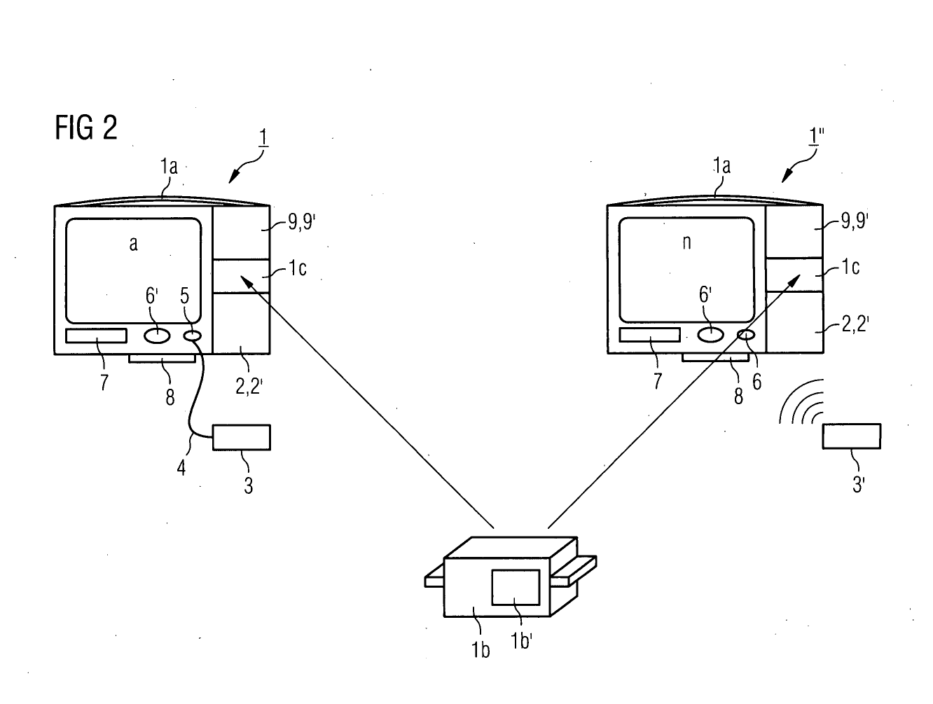

As an alternative embodiment, FIG. 2 illustrates the modular construction and modular use of the mini-laboratory analysis unit 1b integrated into an emergency combi-set 1 or 1″ described above. As can be seen from FIG. 2, the mini-laboratory analysis unit 1b can, for example, be removed from a housing 1c of a first emergency combi-set 1 with just a few simple actions and slid into an empty housing 1c provided for the purpose in a second emergency combi-set 1″

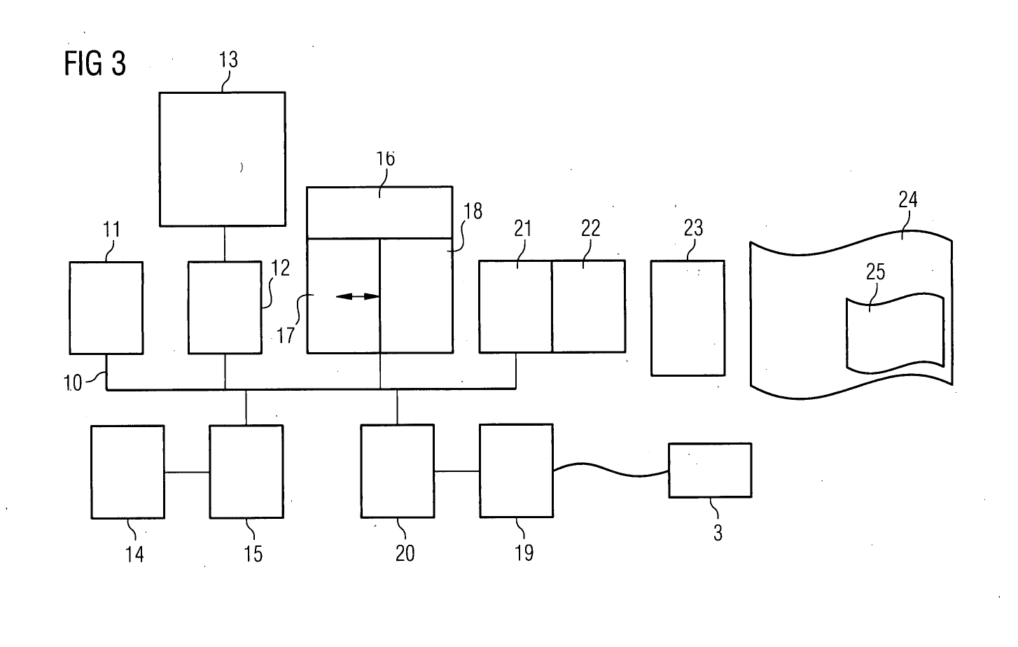

FIG. 3 is a system overview of the system components of one of the emergency combi-sets 1, 1′ or 1″ according to the invention, with an integrated patient monitor, integrated sonographic device and integrated mini-laboratory analysis unit. As can be seen in FIG. 3, the patient monitors of emergency combi-sets 1, 1′ or 1″ are controlled via a data bus 10, to which a microprocessor 11 is connected for control of all the system components and their processes connected to the data bus 10. By means of a patient monitor controller 12, a display 13 with patient monitor functions is connected to the bus 10 on which, for example, curves, data and ultrasonic images of an emergency patient can be shown. By means of a sensor/data interface 14 for the patient monitoring, data from sensors applied to the patient can be sent to a signal processing unit 15, which is connected to the data bus 10, for processing received sensor data. By means of an input/output interface 16 provided for the input and/or output of user data, input menus 17 for patient monitoring and input menus 18 for an ultrasonic signal processing unit 20 can be called up, with the instructions from the input menus being sent via the data bus 10 to the corresponding device. The ultrasonic transducer 3 is connected via an ultrasonic data/interface 19 to the ultrasonic signal processing unit 20 connected to the data bus 10. A memory 21 with a memory expansion 22 for the ultrasonic components is also connected to the data bus 10. Furthermore, the patient monitor of the emergency combi-set 1 is provided with a power supply unit 23 for the power supply of its individual system components. The processes in the patient monitor are controlled by system software 24 provided for this patient monitor, which is expanded by a system software 25 for the ultrasonic signal processing unit 20. An ECG unit, a ventilator and/or resuscitation device (defibrillator) can also be provided.

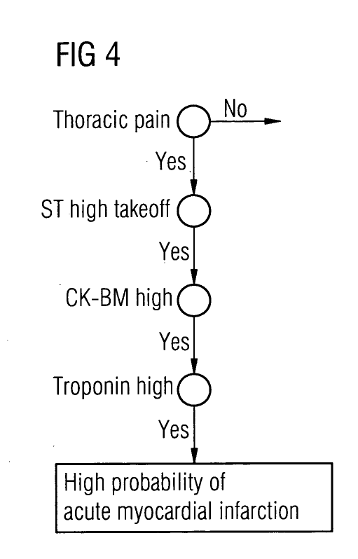

FIG. 4 shows an example of a diagnostic aid consisting of a decision tree for linking acquired medical information, resulting from a combination of the analysis data received with the aid of the integrated mini-laboratory analysis unit 1b. As can be seen from this illustration, the occurrence of an acute myocardial infarction in the near future can be assumed with a high degree of probability only from a parameter combination resulting from a combination of four different medical parameters (thoracic pain, ST high take-off in the ECG, high CK-BM level and high troponin level). Other symptoms can also only be reliably verified if there is a presence of a certain combination of parameters.

As follows from the preceding description, the advantage of the inventive solution lies in the possibility of a distinctly better diagnosis immediately at the emergency site or the “point of care” and the resulting faster introduction of the correct therapeutic measures by emergency medical personnel.

1.-15. (canceled)

16. An examination unit, comprising:
an integrated mini-laboratory analysis unit for a biochemical and/or cell-biology investigation of blood samples or tissue biopsies of a patient; and
at least one device selected from the group consisting of patient monitor, ultrasonic device, ECG unit, ventilator device and resuscitation device.

17. The examination unit as claimed in claim 16, wherein the examination unit is configured as an emergency combi-set for at least one action selected from the group of producing a first diagnosis, monitoring a vital function, initiation of life-supporting, and immediate therapeutic measures for emergency patients with acute cardiovascular diseases, myocardial infarction, stroke or diabetes mellitus symptoms.

18. The examination unit as claimed in claim 17, wherein the emergency combi-set comprises a stationary component selected from the group consisting of “all-in-one” ultrasonic device, patient monitor, ECG, ventilator and resuscitation device.

19. The examination unit as claimed in claim 17, wherein the emergency combi-set comprises a portable component selected from the group consisting of “all-in-one” ultrasonic device, patient monitor, ECG, ventilator and resuscitation device.

20. The examination unit as claimed in claim 19, wherein the portable emergency combi-set is fitted with an integrated mobile power supply unit in the form of a battery, an accumulator, a fuel cell or a solar panel as a power-supply.

21. The examination unit as claimed in claim 16, wherein the integrated mini-laboratory analysis unit provides a capillary electrophoresis.

22. The examination unit as claimed in claim 16, wherein the integrated mini-laboratory analysis unit is suitably provides gene tests for the identification of high-risk patients with respect to a genetic predisposition to the occurrence of a disease.

23. The examination unit as claimed in claim 16, wherein the disease pertains to a cardiovascular disease, a myocardial infarction, angina, an apoplectic stroke, arteriosclerosis or diabetes mellitus.

24. The examination unit as claimed in one of claims 18, further comprises a permanently-integrated ultrasonic unit used only by the emergency combi-set itself.

25. The examination unit as claimed in one of claims 18, further comprises an ultrasonic unit which is integrated, but constructed as an independent module, which can be used both by the emergency combi-set itself or by external technical medical equipment.

26. The examination unit as claimed in one of claims 18, further comprises a connecting socket for plugging in a connecting cable to a connectable external ultrasonic transducer.

27. The examination unit as claimed in one of claims 18, further comprises a reception part for the wireless reception of ultrasonic signals from an external ultrasonic transducer via a wireless interface.

28. The examination unit as claimed in claim 27, wherein the wireless interface is an infrared interface.

29. The examination unit as claimed in claim 27, wherein the wireless interface is a radio interface.

30. The examination unit as claimed in claim 16, further comprises an integrated radio signal transmission unit for the wireless transmission of recorded patient data to a hospital receiving the emergency patient.

31. The examination unit as claimed in claim 16, further comprises an integrated display device for the display of recorded patient data.
